(19) United States
(12) Patent Application Publication
(10) Pub. No.: US 2011/0237994 A1
(43) Pub. Date: Sep. 29, 2011

(54) VOID-FILLING WOUND DRESSING

(75) Inventors: Corey Russ, Fayetteville, NC (US); Christopher Murphy, Vass, NC (US)

(73) Assignee: COMBAT MEDICAL SYSTEMS, LLC, Fayetteville, NC (US)

(21) Appl. No.: 12/732,056

(22) Filed: Mar. 25, 2010

(51) Int. Cl. A61F 13/00; A61L 15/44

(52) U.S. Cl. 602/46; 602/43; 602/48; 602/53

(57) ABSTRACT

A wound dressing includes a wound-filling member attached to a hemostasis inducer that can be impregnated with an active compound, such as coagulants. The wound dressing can be placed in wounds to provide pressure, as well as chemical action, to promote blood coagulation and thereby arrest bleeding. A wrap can be attached to the wound dressing to provide an easy and quick method of securing the wound dressing. The wound dressing can be compressed in a small package for easy transport.

### TECHNICAL FIELD

The present invention relates generally to medical wound dressings, and more specifically to wound dressings positioned at least partially inside a wound.

### BACKGROUND

Wound dressings, particularly hemostatic dressings, have significantly contributed to increased survivability from physical trauma. As used herein, wound dressing refers generally to generally flexible materials, such as gauzes, bandages, fabrics, etc., that can be used to treat recent wounds. Wound dressings can help to control bleeding and protect the wound from entry of bacteria or other infective agents. In combat situations, wounds are often life-threatening and the time pressures always associated with treating wounds may be amplified by ongoing hostilities. Especially prevalent on the modern combat battlefield are large wounds that can be caused by explosives and blunt force trauma. For example, modern soldiers risk injuries from Improvised Explosive Devices (IEDs), which often cause large soft tissue defects.

Treating a large wound is challenging due in part to the large surface area of the wound. In some instances, the large soft tissue defect can comprise a cavity where a portion of the patient's body has been separated or removed. It can be very difficult to attempt to stanch bleeding in a large-cavity wound.

In extreme situations, a tourniquet can be applied to a large wound to prevent major blood loss. However, a disadvantage of applying a tourniquet is that a substantial amount of undamaged issue may be deprived of circulation and necrotize, broadening corporeal damage beyond the original wound and significantly increasing the risk of gangrene.

### SUMMARY OF THE DISCLOSURE

In some embodiments, the wound dressing is a compact, easily deployable device or agent that addresses hemostasis in large soft tissue defects. It can provide a simple-to-use, easily carried, integrated system for arresting bleeding and suppressing or providing an impediment to infection at a traumatic injury site. In some embodiments, a wound dressing can be applied to a wound to rapidly arrest bleeding by generally filling a wound cavity, or at least a portion thereof, with a filler material, such as a compressible and/or expandable foam, to form a physical barrier and to provide moderate compression against the interior of the wound cavity. The filler material and/or other components of the wound dressing can also create a physical and chemical barrier against undesirable foreign objects such as dirt and bacteria to fend off infection.

In some embodiments, the filler material can be permanently attached to a contact layer on one or more surfaces thereof. Either or both of the contact layer and the filler material can include therapeutic agents, such as hemostatic agents and anti-microbial agents, to help in treating the wound.

### BRIEF DESCRIPTION OF THE DRAWINGS

FIG. 1 is a top plan view of a wound dressing according to an embodiment of the invention.

FIG. 2 is a side elevational view of the wound dressing of FIG. 1.

FIG. 3 is a schematic top view of the wound dressing of FIG. 1 positioned in a wound cavity on a leg of a patient.

FIG. 4 is a schematic top view similar to the view of FIG. 3 illustrating the wound dressing expanded in the wound cavity and an elastic wrap around the patient's leg.

FIG. 5 shows a cross-sectional view of FIG. 4 taken at 5-5.

### DETAILED DESCRIPTION OF SPECIFIC EMBODIMENTS

In some embodiments, the wound dressings described herein can comprise a conformable wound-filling material, such as foam, that is secured to a hemostatic pad. The dressings can be placed in the cavity of large soft tissue defects. The wound dressings can provide and maintain effective temporary hemostasis when applied to a wound to stabilize the patient until further medical treatment can be received. Effective hemostasis, as used herein, is the ability to control and/or decrease bleeding within a therapeutically effective time, as recognized by those skilled in the art of hemostasis. As used herein, unless otherwise specified, the terms “fill,” “filler,” and “filling,” and other variants thereof, refer to something that is positioned at least partially inside of something else to eliminate at least a significant portion of a void, but these terms should not be construed to require that the void is entirely eliminated.

Wound dressings, as disclosed herein, can be useful in many circumstances. In some embodiments, the disclosed wound dressing can be particularly advantageous for treating large battlefield wounds where compactly packaged, quickly deployable bandages are beneficial. Although described in the context of trauma wounds, in other circumstances, wound dressing can be used to treat other wounds, such as decubitus ulcers, pressure sores, venous stasis ulcers, infected wounds, deep and open surgical incisions and burns, and other situations, where keeping the wound clean and free from infection is advantageous.

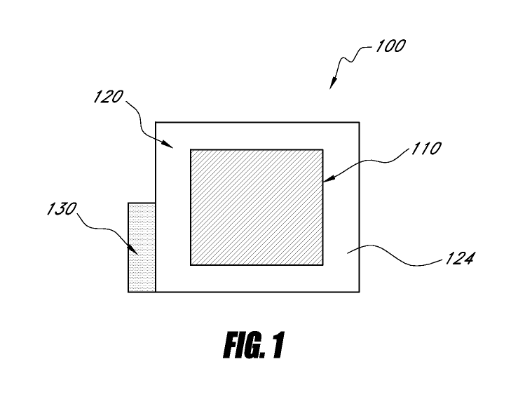

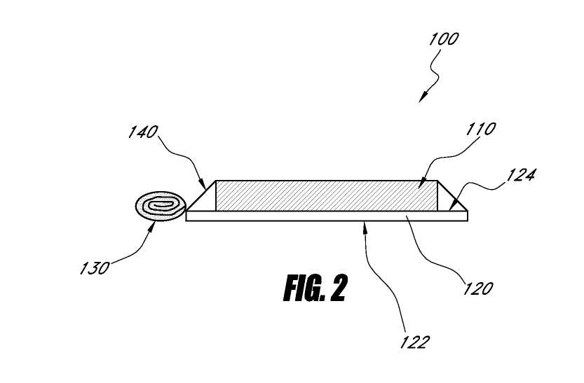

FIG. 1 illustrates a wound dressing 100 according to some embodiments of the invention. In the illustrated embodiment, the wound dressing 100 comprises a wound-filling member 110 attached to a hemostasis inducer 120. Attached to a side of the hemostasis inducer 120 can be a wrap 130. In some embodiments, as illustrated in FIG. 2, a cover 140 can be attached to the hemostasis inducer 120 and wrap around the wound-filling member 110.

In some embodiments, the wound dressing 100 can be used to treat a wound by placing the hemostasis inducer 120 against the wound surface with the wound-filling member 110 attached to a side of the hemostasis inducer 120 opposite from the wound facing side. After the hemostasis inducer 120 and the wound-filling member 110 are in position, the wrap 130 can surround the patient and the wound dressing 100 at the wound site to secure the wound dressing 100 to the patient and to help provide pressure against the wound to assist in stanching blood flow.

In some embodiments, the wound dressing 100 can be placed in a wound cavity. The wound-filling member 100, or at least a portion thereof, can generally fill the wound cavity to form a physical barrier and to provide moderate compression against the wound cavity. In some embodiments, the wound-filling member 100 can be compressed prior to being placed at least partially within the wound cavity, where it can then expand against at least some of the cavity walls.

In some embodiments the wound dressing 100 can be applied to a wound that does not have a cavity or has a small cavity. The hemostasis inducer 120 can lie on top of the wound and the wound-filling member 110 can lie on top of the hemostasis inducer 120. In some embodiments, the wound-filling member 110 can be generally resilient, compressible and/or expandable. When the wrap 130 is secured over the hemostasis inducer 120 and the wound-filling member 110, the wound-filling member 110 can expand and help to create pressure against the wound to encourage hemostasis.

In some embodiments, the wound-filling member 110 comprises foam or foam-like properties. In some embodiments, the wound-filling member can be a compressible foam. In some embodiments, the wound-filling member 110 can be any resilient material, such as a sponge-like or rubber-like material.

In some embodiments, the wound-filling member 110 can be compressible while having some degree of resiliency or rigidity. The wound-filling member 110 can be compliant and can conform, at least partially, to the contours of a wound when placed in a wound cavity. Thus, the wound-filling member 110 can have sufficient resiliency to tend to forcefully rebound to its natural shape, which in some embodiments is generally flat. In some embodiments, the wound-filling member 110 is sufficiently flexible to adjust to the patient's body contours and can adjust to the patient's body even when the patient is in movement. In some embodiments, as illustrated, the wound-filling material 110 can be an integrated, one-piece member without seams, spaces, or gaps between individual portions or pieces thereof. Such integral wound-fillers can result in a more forcefully expansive wound dressing 100 that provides increased internal pressure against the inside of a wound after it is deployed. In other embodiments, the wound-filling member 110 can be comprised of discrete portions capable of moving around within an enclosure to more closely conform to the shape of a wound cavity.

In some embodiments, the wound-filling member 110 and the wound dressing 100 can be packaged into a generally small form to permit convenient carrying and storage, especially in the limited space available to combat medics or other medical personnel. In some embodiments, the compressible nature of the material enables the wound-filling member 110 to be at least partially packed or stuffed into a wound cavity. In some embodiments, the material used in the wound dressings 100 can have sufficient compressibility to enable the wound-filling member 110 to be compressed and then expand to conform, at least partially, to the contours of the wound, thereby exerting an outwardly directed pressure against the wound surfaces and promoting hemostasis. In some embodiments, an example of a useful type of filler material for the wound-filling member 110 will permit the following compression under these test circumstances: a 10 cm by 10 cm cube of material when subject to an added pressure of 1 Kg/0.1 m² can collapse to less than or equal to about 10% and preferably to less than or equal to about 5% of its uncompressed volume, and then approximately return to its original, uncompressed volume when the added pressure is removed.

In some embodiments, the wound-filling member 110 can recover completely or virtually completely to its uncompressed configuration when the pressure is removed. When placed in the wound cavity, the wound-filling member 110 can expand until it contacts at least a portion of the cavity wall and provide pressure on the wall to promote hemostasis. The ability of the wound-filling member 110 to return to its undisturbed or original size and shape can be measured through several methods, such as using a compression set test. In a compression set test, a test sample is typically compressed in a particular direction by a specified distance, and then the compression force is withdrawn and the sample is permitted to rebound. The compression set test value is a measure of the percent of the compression distance that a wound-filling member 110 does not recover after being compressed. A more specific type of compression set test can determine the amount by which a wound-filling member does not recover after being compressed by a specified percentage amount. For example, a 90% compression set test measures the rebound of the wound-filling member 110 after being compressed to 90% of its uncompressed size. For instance, in some embodiments, the wound-filling member 110 can have a 90% compression set rating of approximately 10% or less. This means that after the wound-filling member 110 is compressed by 90% of its uncompressed size, the wound-filling member 110 will rebound until it has recovered all but approximately 10% or less of the distance by which the material was compressed. In some embodiments, the wound-filling member 110 can recover more fully and the 90% compression set rating can be less than 10% (e.g., less than about 8%). In other embodiments, the wound-filling member 110 may not recover as much and the 90% compression set rating can be greater than 10%.

The amount of time needed for the wound-filling member 110 to rebound to its original size and shape or to its modified size and shape (for materials that do not fully rebound) after compression can affect the performance of the product. For example, it can be beneficial to have a shorter recovery time after compression so that the wound-filling member 110 quickly fills the wound cavity, in use. In some embodiments, the recovery time can range from at least approximately 1 second and/or less than approximately 10 seconds. In some embodiments, the compression recovery time can be greater than approximately 10 seconds. In some embodiments, the recovery time can be less than about 1 second, and in other embodiments the recovery time can be less than approximately 5 seconds.

In some embodiments, the wound-filling member 110 has sufficient resilience to provide therapeutic pressure against the wound cavity as it expands within the wound cavity. The resilience can be determined in many ways. An example of a resilience test involves dropping a steel ball from a specified height onto the wound-filling member 110 and recording the rebound height of the ball. The value is recorded as a percentage of the original drop height. For example, the wound-filling member 110 can be configured to be resilient and somewhat rigid with a resilience of at least approximately 45%. In some embodiments, the wound-filling member 110 can be softer, with a resilience of less than approximately 45%. In some embodiments, the wound-filling member is semi-rigid and resilient, and can have a resilience ranging from at least approximately 25% and/or less than approximately 75%.

In some embodiments, the wound-filling member 110 can be capable of absorbing fluids from the wound. For example, the wound-filling member 110 can have the capacity to absorb several times its own weight in fluids, such as blood, serum, or other wound exudate. In some embodiments, the wound-filling member 110 can absorb at least three times its weight in body fluid and preferably at least four times its weight of body fluid. In some embodiments, the wound-filling member 110 can generally maintain its shape and size upon absorbing the wound exudate.

In some embodiments, the wound-filling member 110 can swell upon absorbing the wound exudate. In some embodiments, the wound-filling member 110 can swell and continue to be generally conformable and/or resilient. In embodiments where the wound-filling member 110 swells, the wound-filling member 110 can apply increasing pressures against the walls of a wound cavity as it swells. The increased pressures from the swollen wound-filling member 110 can help arrest the bleeding from the wound and provide hemostasis.

Some absorbent wound-filling members 110 can provide a wound dressing 100 that absorbs exudate quickly and maintains the wound surface in a moist condition. A moist wound surface can encourage healing of the wound and prevent undesired adhering of the dressing to the wound. In some situations, it can be desirable to conduct the wound secretion away from the wound site so that the wound secretion does not coagulate. This can be desirable to prevent the formation of an impenetrable crust over the wound, which in some situations can lead to pooling of further exudate under the crust and delaying wound healing or causing infection.

As discussed earlier, the wound-filling member 110 can comprise a compressible, expandable, and compliant material, such as foam. Some examples of foams include polyurethane, carboxylated butadiene-styrene rubber, polyacrylate, polyvinylic or cellulosic foams. Polyvinylic foams include polyvinyl acetal foams formed by the reaction of polyvinyl alcohol and an aldehyde, usually formaldehyde or acetaldehyde. Such foams are generally more flexible when wetted by water. It is envisaged that such foams can be used dry or can be wetted and squeezed dry whereupon they retain sufficient moisture to remain soft and flexible and can be packaged in a waterproof package.

In some embodiments, the material of the wound-filling member 110 can be made from hydrophilic materials or can be treated to render them hydrophilic, for example with surfactants. Hydrophilic foams can be less likely to cause the wound exudate to coagulate rapidly, which again can encourage healing of the wound and prevent undesired adhering of the dressing to the wound. Hydrophilic polyurethane foam is an example of a suitable foam. In some embodiments, the polymer foams can be made of crosslinked hydrophilic polyurethane. In some embodiments, the polymer foams can be made by reacting a hydrophilic isocyanate-terminated polyether prepolymer with water.

In some embodiments, the wound-filling member 110 can be formed by foaming, for example by blowing or reticulating hydrophilic polyurethane. Suitable polyurethanes can include linear polyether polyurethanes. The hydrophilic polyurethanes can be foamed using a conventional blowing agent or can be reticulated by conventional means. In some embodiments, the foam can be cast in a foamable composition onto a support to which it is not adherent and after curing the wound-filling member 110 can be recovered in the form of a sheet having a desired thickness. In some embodiments, the wound-filling member 110 is manufactured separately and exists as a separate resilient component before being attached to other layers of the wound dressing 100.

In some embodiments, the wound filling member 110 can be a polyurethane foam of grade 1240. In some embodiments, the polyurethane foam can have one or more of the following characteristics when tested under ASTM D-3574-91 standards: density of approximately at least 1.15 pounds per cubic foot; resilience (as defined above) of approximately 45% or more; tensile strength of approximately 15 pounds per square inch or more; elongation of approximately at least 150% of normal length; tear strength of approximately at least 1.5 pounds per inch; and 90% compression set (as defined above) of approximately 10% or less.

In some embodiments, a blend of two or more foams can be used or a combination of a foam with an absorption enhancing material such as, for example, a cellulose material in a form which is capable of being packaged within the bag.

In some embodiments, the wound-filling member is a generally flat and generally square or rectangular block. For example, the size of the wound-filling member 110 can be approximately 15 cm in width and approximately 15 cm in length. The thickness of the material can be approximately 5 cm. In some embodiments, the width and length can each be at least approximately 10 cm and/or less than or equal to approximately 20 cm. In some embodiments, the thickness can be at least approximately 2 cm and/or less than or equal to approximately 10 cm.

In some embodiments, the wound-filling member 110 can be a resilient, open cell foam. In some embodiments, the open cell foams can have cell diameter sizes of at least approximately 30 μm and/or less than or equal to approximately 700 μm, and preferably cell diameter sizes of at least approximately 50 μm and/or less than or equal to approximately 500 μm. In some embodiments, the total volume of the open cell foams be at least about approximately 20% and/or less than or equal to approximately 70% of open cells. In some embodiments, the volume of the open cell foams can comprise a range of at least approximately 30% and/or less than or equal to approximately 60% of open cells. Such open cell foams can allow absorption of fluids and wound exudate secreted from the wound into the foam structure.

In some embodiments, the foam can be a resilient, closed cell foam. The closed cell foam can advantageously comprise generally uninterrupted surfaces, which can provide flat pressure to the hemostasis inducer 120 to press against the walls of the wound. In some embodiments, the uninterrupted surface of the closed cell foam can act as barriers to stop blood or bodily fluids from escaping the patient's body and promoting clotting of the blood. In some embodiments, the closed-cell foam can be non-absorbent. The material of the foam can be selected so as not to disperse, disintegrate, or dissolve when it comes in contact with fluids such as water and blood.

In some embodiments, the wound-filling member 110 may not contain any active compounds. Unlike the hemostasis inducer 120, which in some embodiments can contain physiologically active components, such as organic or inorganic coagulant inducers, antibacterial agents and local anesthetics, the wound-filling member 110 may not contain active compounds and can be configured not to directly contact the wound.

In some embodiments, the hemostasis inducer 120 can be a relatively thin layer or pad, such as a gauze, that can be placed over a wound in a manner that provides direct contact with the wound. The hemostasis inducer 120, as well as any other components or the wound dressing 100, can be sterile so that it does not introduce contaminants or bacteria to the wound. In some embodiments, the hemostasis inducer 120 can be absorbent and draw fluids away from the wound.

In some embodiments, the hemostasis inducer 120, and/or the wound-filling member 110, or other components of the dressing 100, can comprise hemostatic agents that are applicable to bleeding wounds to promote hemostasis, such as disclosed in U.S. Pat. No. 7,604,819, issued Oct. 20, 2009 and incorporated herein by reference in its entirety. The TraumaPad™, sold by Z-Medica Corporation of Wallingford, Conn., is one example of a suitable hemostasis inducer 120. The hemostasis inducer 120 can provide a backing to which the wound-filling member 110 can be attached, either directly or indirectly. In some embodiments, the wound-filling member 110 can be permanently attached, such as by sewing it into the center of the hemostasis inducer 120. Many advantages can be achieved with a generally central placement of the wound-filling material 110 on the hemostasis inducer 120 in which a perimeter of the hemostasis inducer 120 generally surrounds the wound-filling material 110, as illustrated. For example, in this configuration, at least a portion of the perimeter region of the hemostasis inducer 120 can be free to fold up or otherwise flex independent of the wound-filling material 110 during deployment of the dressing 100 into or around a wound. In this way, the perimeter region can generally cover or be pushed against the sides of at least a portion of the wound-filling material 110. The dressing 100 can thereby present a hemostatic surface around the sides of the wound-filling material 110 and generally avoid direct contact between the wound-filling material and the internal surface of the wound. In some embodiments, the wound-filling member 110 can be attached to the hemostasis inducer 120 through other means, such as adhesive bonding. In some embodiments, the foam may not be attached to the hemostasis inducer 120 and can be held in place by a retainer, such as a mesh cover 140.

In some embodiments, the hemostasis inducer 120 can be disposed on both sides of the wound-filling material 110. In some embodiments, the hemostasis inducer 120 can form a pouch such that the wound-filling material 110 is generally encased by the hemostasis inducer 120. The dressing 100 can thus be configured so that it can be deployed by placing either of its sides against the wound. This configuration can be useful in preventing the dressing 100 from being incorrectly deployed with the wound-filling material placed directly against the wound instead of the hemostasis inducer 120. This configuration can also facilitate easier deployment under low light levels, and quicker deployment, especially in stressful combat environments.

The hemostasis inducer 120 can be any suitable woven or non-woven fibrous material including, but not limited to, cotton, silk, wool, plastic, cellulose, rayon, polyester, combinations of the foregoing, or other materials. The hemostasis inducer 120 is not limited to woven or non-woven fibrous materials, however, as felts and other materials can be used. In some embodiments, the hemostasis inducer 120 can be a sponge or sponge-like material. In some embodiments, the hemostatic agents can include clay materials or other silica-based materials.

The term “clay” refers to a crystalline form of hydrated aluminum silicate. In some embodiments, the crystals of clay can be irregularly shaped and insoluble in water. The combination of some types of clay with water can produce a mass having some degree of plasticity. Depending upon the type of clay, the combination thereof with water can produce a colloidal gel having thixotropic properties.

In some embodiments, the clay material can comprise kaolin, which includes the mineral “kaolinite.” Although the term “kaolin” is used hereinafter to describe the hemostatic agents, it should be understood that kaolinite can be used in conjunction with other materials. The hemostatic agent is also not limited to kaolin or kaolinite. In some embodiments, the hemostasis inducer 120 can include materials such as attapulgite, bentonite, combinations of the foregoing, combinations of the foregoing with kaolin and/or diatomaceous earth, and the like.

In some embodiments, the kaolin coating can be deposited on the substrate by applying the kaolin in slurry form on one side of the hemostasis inducer 120 using a spraying technique, a slot die technique, or a combination thereof. In using any technique, the amount of slurry applied to the hemostasis inducer 120 can be limited to avoid or at least minimize the saturation of the substrate. A colloidal form of the kaolin (or other clay) can be used to provide a stable suspension of the material with suitable viscosity for application using the slot die technique. Once sprayed or applied using the slot die technique, the coated hemostasis inducer 120 can be rolled or scraped to further embed the kaolin into the material of the substrate. The hemostasis inducer 120 can then be dried.

In some embodiments, a release agent can be disposed on the wound-contacting side 122 of the hemostasis inducer 120, or on any other suitable portion of the dressing, to facilitate the easy removal of the hemostasis inducer 120 from the wound tissue after the formation of blood clots. The release agent may be a continuous film, or it can be discontinuous on the surface of the substrate.

In some embodiments, the release agent can be applied to the non-wound contacting side 124 of the hemostasis inducer 120 as a slurry of clay and release agent. For example, a concentration of polyvinyl alcohol or glycerol can be applied such that at least some of the alcohol component thereof seeps to the wound-contacting surface of the hemostasis inducer 120, while the clay material remains on or near the non-wound contacting surface. In any embodiment, the polyvinyl alcohol or the glycerol serves not only as a release agent, but as an agent that suppresses the dust of the particled kaolin.

Other materials that can be used as release agents include, but are not limited to, silicone and gelatinized starches. As with polyvinyl alcohol and glycerol, either can be applied in film form.

The hemostasis inducer 120 can be sufficiently flexible, even after application of the hemostatic agents, to allow it to be folded, rolled, or otherwise manipulated for packaging. The flexibility of the hemostasis inducer 120 also allows the hemostasis inducer 120 to conform to the contours of the wound as the wound-filling member 110 is placed in the wound and expands into the shape of the wound, pressing the hemostasis inducer 120 against the wound cavity.

A cover 140 can be wrapped around the front, back (as illustrated in FIG. 2), or non-wound contacting side 124 of the hemostasis inducer 120, which can also enclose the wound-filling member 110. In some embodiments, the material that forms the cover 140 can be flexible and elastic. In some embodiments, as the wound-filling member 110 within the cover 140 unfolds or otherwise expands on absorbing wound exudate, the cover 140 is capable of extending to compensate for the increased volume of the wound-filling member 110 while not tearing under the increased strain. The material of the cover 140 can be flexible so that it is conformable to the irregular contours of the wound cavity.

In some embodiments, the cover 140 can have a soft feel when in contact with the skin and can be liquid permeable by virtue of absorption and/or passing through apertures through the cover 140, for example in a mesh or net structure. By liquid permeable, it is meant that the cover 140 can be adapted to allow the passage of liquids such as blood, water, and wound exudate. The material can possess apertures by virtue of weaving or some other manufacturing process, such as an integral net. In some embodiments, the apertures can be formed in a film of the material by means of conventional methods including needling, electric discharge, vacuum perforation, hot jet perforation and moulding under heat and pressure on a suitable former or by fibrillation of an embossed film. In some embodiments, the cover 140 can be made from hydrophobic polymers including high or low density polyethylene, polypropylene, polyurethane, polystyrene or copolymers or mixtures thereof or styrene-butadiene or styrene-isoprene-foam member copolymers.

In some embodiments, the cover 140 can be sewn to the back of the hemostasis inducer 120. In some embodiments, the cover 140 can be attached through other means, such as adhesives, chemical bonding, or by being integrally formed with the hemostasis inducer 120, or by forming the covering as a casing into which the hemostasis inducer 120 and wound-filling member 110 are inserted. In some embodiments, the cover 140 is not fixed to the wound-filling member 110, which can advantageously allow the wound-filling member 110 to expand, contract and move relative to the cover 140. In some embodiments, the cover 140 can be adhered to the wound-filling member 110 through any attachment means, such as discussed earlier.

The apertures in the cover 140 can be any suitable shape, such as a generally circular shape. In some embodiments, the average size of the apertures can be at least approximately 0.1 mm and/or less than or equal to approximately 4 mm in diameter, and preferably at least approximately 1 mm and/or less than or equal to approximately 2.5 mm in diameter. In some embodiments, the apertures can have irregular shapes, in which case they can have an area equivalent to that of the circular apertures described above. In some embodiments, the contoured net can have a thickness of at least approximately 0.5 and/or less than or equal to approximately 2 mm, and preferably at least approximately 1.0 and/or less than or equal to approximately 1.5 mm.

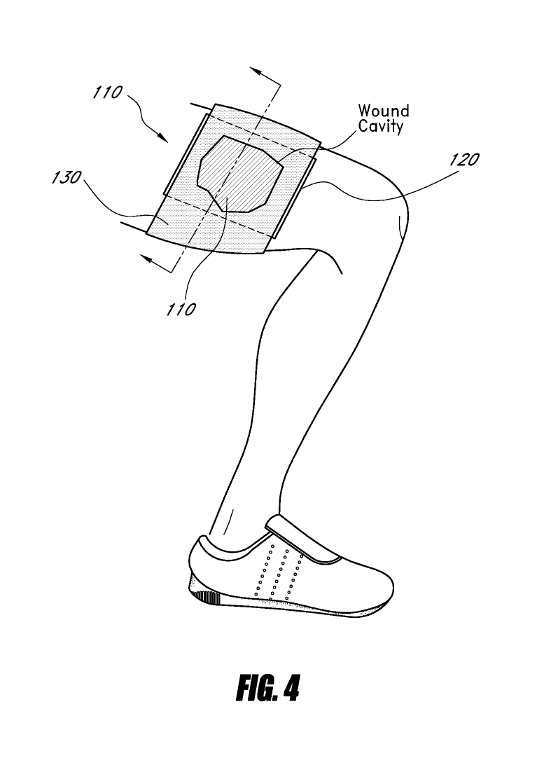

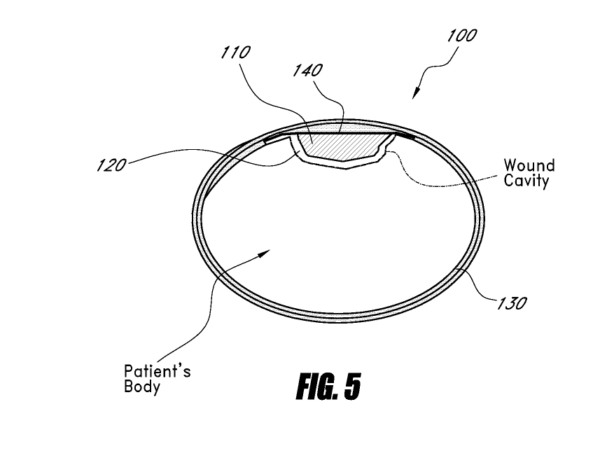

In some embodiments, the wound dressing 100 can be held in place by adhesive bandages or elongate pieces of material wrapped around the wound dressing 100. In some embodiments, the wound dressing 100 can be held on the wound by wrapping a wrap 130 around the patient's body as illustrated in FIGS. 4 and 5. In some embodiments, the wrap 130 is an elongate piece of material, such as gauzes, bandages, fabrics, etc. In some embodiments, the wrap 130 can be elastic and able to stretch around the wound site.

In some embodiments, as illustrated in FIGS. 1-3, the wrap 130 can be attached to the wound dressing 100 for easier deployment. In some embodiments, the wrap 130 can be sewn to an edge of the hemostasis inducer 120. In some embodiments, the wrap 130 can be attached through any method, such as adhesives or rivets. In some embodiments, the wrap 130 can be separate from the wound dressing 100, but preferably included in the same package. As illustrated, the wrap 130 initially can be provided in a generally tight roll, which diminishes its storage profile and facilitates the positioning of the wrap around the wound in an unrolling process.

In some embodiments, the wrap 130 can be approximately 15 cm in width and approximately 122 cm in length. In some embodiments, the wrap 130 can have a width of at least approximately 5 cm and/or less than or equal to approximately 21 cm, and the length can be at least approximately 60 cm and/or less than or equal to approximately 183 cm.

Once the wrap 130 is wrapped around the patient, it can be secured by any means known in the art to prevent the wrap 130 from unraveling, such as hooks or adhesive tapes. In some embodiments, the hooks, adhesive tapes or other securing means can be pre-attached or integrated to the wrap 130 for easier and quicker deployment of the wound dressing 100. In some embodiments, the wrap 130 is made of a material that is cohesive or self-adherent, so that the wrap 130 can adhere to itself as it is wrapped around the patient. A self-adherent wrap 130 can remove the need to have a separate attaching means, which can reduce the packaged size of the wound dressing 100.

In some embodiments, the wrap 130 can be made of a porous, water and blood permeable material. In some embodiments, additional wound dressings 100 can be placed on top of the first wound dressing 100 in situations where additional absorption is necessary. In some embodiments, the wrap 130 can be made of an impermeable material, such as plastics or treated fabrics, to protect the wound dressing 100 and wound from dirt, liquids or other contaminants.

The wound dressing can be in any convenient form suitable for a wound. In some embodiments, the wound dressing can be in the form of a generally square shape. In some embodiments, the dressing can be in the form of an approximate circle or an approximate oval. The size and shape of the wound dressings 100 can depend on the type of wound to which the wound dressing is to be applied. For example, a large wound caused by an explosion or large caliber bullet can require dressings which are at least about 30 cm by 30 cm in a substantially square shape, or can require the use of multiple smaller wound dressings.

In some embodiments, the wound dressing can comprise an elongate hemostasis inducer in which the ratio of length to width can be about 10:1 or greater. The wound dressing can include multiple pouches formed by a cover attached to the hemostasis inducer, into which the wound-filling member can be inserted. The wound dressing can be employed in a variety of ways. For example, in some embodiments, the wound dressing can be used as a single dressing by inserting the wound-filling member into a single pouch. The single filled pouch can be packed into a wound and the elongate hemostasis inducer can be used to wrap around the patient and secure the wound dressing. In another example, several wound-filling members can be inserted into multiple pouches to make several dressings from which individual dressings can be cut. In another example, a combination of two or more pouches can be filled with wound-filling members and used to treat an extra large wound cavity.

The shape and size of the wound dressing can be selected to most effectively treat a particular wound. The size and shape of the wound dressing can be large enough for the wound-filling member to at least partially fill the wound cavity and provide pressure against at least portions of the walls of the wound cavity, while not being overly large such that the pressure exerted on the wound becomes overly difficult to insert, injurious to the wound, or excessively uncomfortable to the patient or disruptive to the healing process.

In some embodiments, the wound dressing can be compressed and wrapped in a compact package. As described above, the compressible nature of the wound-filling member can help in producing a package that takes up minimal space. This can be desirable in many situations, such as combat environments where soldiers must carry the wound dressings during missions, along with large amounts of other equipment. The compact nature of the device can be especially useful for medics, whose combat lifesaver bags are already filled with other equipment and fluids. Thus, the compact and lightweight qualities of the packaged wound dressing disclosed herein are particularly advantageous for use by military personnel. However, the wound dressing is also advantageous for use outside the military context, such as by first responders whose vehicles have limited space for supplies.

In some embodiments, the wound dressings 100 are sterile and the packaging is preferably provided in microbial resistant pouches. The wound dressing 100 can also be vacuum-packed in its container to diminish its volume and to make it easier to store and to carry. Such packages can be prepared under aseptic conditions or alternatively sterilized after packing by a another procedure, such as for example heat sterilization. In some embodiments, the wound dressings can be sterilized by ethylene oxide or gamma irradiation. In some embodiments, the wound dressings can be heat sealed in packages constructed of aluminum foil laminated with a heat sealable polymeric film such as polyethylene and sterilized in the package by irradiation. In some embodiments, the wound dressings can be sterilized and then sealed in a plastic package. For example, in some embodiments, the wound dressing can be placed in a package and then vacuum sealed. This method of package advantageously compresses the wound dressing and can also create a sterile, impervious pouch. In some embodiments, the packaging material is constructed of durable materials that can withstand abrasion and harsh environments. In some embodiments, the packaging can be submersible and constructed of multiple layers of materials.

In some embodiments, the packaging can be designated with special markings to enable the user to quickly identify the wound dressing. In civilian situations, the packaging can have bright, clearly distinguishable markings to easily find and identify the dressing. Conversely, in combat environments, the packaging can have a drab color and a dull appearance to provide tactical camouflage. In some embodiments, the packaging may not provide tactical camouflage, but can be wrapped with a camouflage enclosure.

To facilitate the opening of the package, the package can have a pre-cut notch or a zig-zag edge pattern where the package can tear easily when a shear force is applied. In some embodiments, the package can be torn without the use of tools and can be opened with the use of only hands.

Many methods can be used to treat a wound with the wound dressing 100. A first responder or medic can identify an injury or wound for receiving a wound dressing 100. In some embodiments, the wound can be cleaned of contaminants and/or sterilized by any known means. In some embodiments, additional drugs, such as coagulants and pain relievers can be applied to the wound.

A wound dressing 100 of appropriate size can be selected and taken out of the medic bag or other carrying bag. The medic can remove the wound dressing 100 from its package by tearing the package open. In some embodiments, the package can be discarded and can be biodegradable. In some embodiments in which the wound dressing 100 was compressed in the package, it can expand once it is removed from the package. The wound dressing 100 can be grasped on the back, or non-wound contacting side 124, and oriented such that the wound contacting side 122 faces the wound.

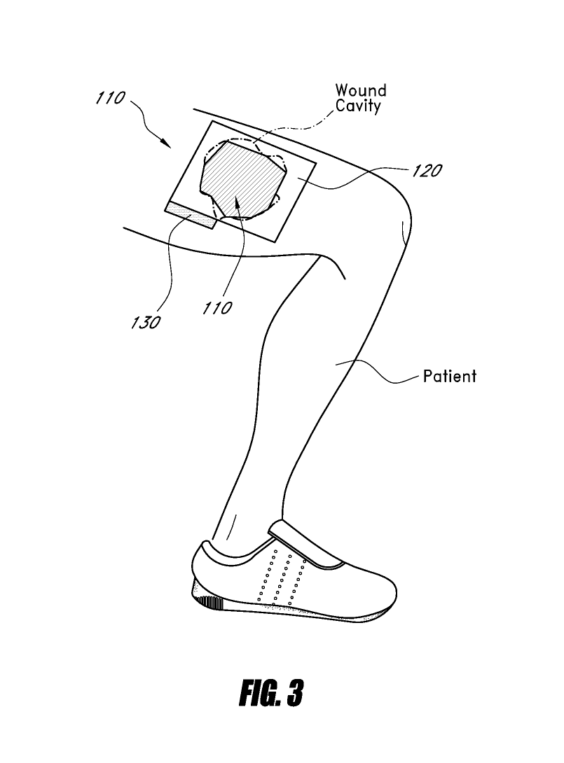

With reference to FIG. 3, the wound dressing 100 can be applied to the cavity of the wound by placing the hemostasis inducer 120 with the active compositions, such as coagulants, against at least portions of the surfaces of the wound. In some embodiments, the natural profile or the wound dressing 100 is substantially larger than the surface opening of the wound on which it is intended to be used. In a deployment mode, at least a portion of the wound dressing 100 comprising the hemostasis inducer 120 and the wound-filling member 110 can be folded over, crumpled up, generally balled up, or otherwise made smaller to compress it and diminish its profile to enable it to be at least partially inserted into the wound cavity. Although the wound dressing 100 is illustrated as applied to a leg in FIG. 3, in some embodiments the wound dressing 100 can be applied to other parts of the body and is not limited to the application illustrated.

In some embodiments, the hemostasis inducer 120 can be positioned at least partially in-between the wound-filling member 110 and the walls of the wound cavity. As the wound-filling member 110 expands or moves outwardly from its position or shape in the deployment mode, it can push some portions of the hemostasis inducer 120 firmly against the walls of the wound cavity. Thus, the wound dressing 100 can be placed into large tissue defects not only to fill the gap created by the trauma, but also to provide advanced hemostasis. In some embodiments, the wound-filling member 110 can be very tightly compressed before being packed into the wound cavity. In some embodiments where the wound is too large to be treated by a single wound dressing 100, multiple wound dressings can be placed in a single wound.

As described above, once the wound dressing 100 is positioned in the wound cavity, the wound-filling member 110 can be allowed to expand, pushing at least some portions of the hemostasis inducer 120 against the walls of the wound, as illustrated in FIGS. 4 and 5. This expansion can increase direct pressure, and can increase conformity and increase direct surface area contact between the internal surface of the wound and the impregnated hemostasis inducer 120, thus providing advanced hemostasis. In some embodiments, the wound-filling member 110 can at least partially conform and create moderate pressure, while not causing tissue damage or creating a tourniquet effect from overpressure. This advantageously can increase hemorrhage control in large soft tissue defects without concerns for further patient injury.

The wrap 130, which can be integrally attached to the wound dressing 100 or can be a separate component, can be unwound and wrapped over the hemostasis inducer 120 and wound-filling member 110 to secure the wound dressing 100 to the wound, as illustrated in FIGS. 4 and 5. As described above, the wrap 130 can be cohesive or self-adherent so that the wrap 130 can be employed without additional tools.

In some embodiments, another wound dressing 100 can be positioned over the first wound dressing 100 in situations where one wound dressing 100 is insufficient to stop the bleeding from the wound. The additional wound dressing 100 can provide increased pressure and more fluid absorption.

Although certain embodiments, features, and examples have been described herein, it will be understood by those skilled in the art that many aspects of the methods and devices illustrated and described in the present disclosure may be differently combined and/or modified to form still further embodiments. For example, it is contemplated that any component of the void-filling wound dressing illustrated and described above can be used alone or with other components. Additionally, it will be recognized that the methods described herein may be practiced in different sequences, and/or with additional devices as desired. Such alternative embodiments and/or uses of the methods and devices described above and obvious modifications and equivalents thereof are intended to be included within the scope of the present invention. Thus, it is intended that the scope of the present invention should not be limited by the particular embodiments described above, but should be determined only by a fair reading of the claims that follow.

The following is claimed:

1. A dressing for treating wounds, the dressing comprising:
a hemostasis inducer with a wound contacting side and a back side;
a wound-filling member configured to conform to the shape of a wound cavity;
a wrap coupled to an edge of the hemostasis inducer;
a cover over the back side of the hemostasis inducer.

2. The dressing of claim 1, wherein the wound-filling member is compressible.

3. The dressing of claim 1, wherein the wrap is elastic.

4. The dressing of claim 1, wherein the cover is made of a mesh material.

5. The dressing of claim 1, wherein the dressing is packaged in a compressed configuration.

6. The dressing of claim 1, wherein the wound-filling member is made of a foam-like material.

7. The dressing of claim 1, wherein the wound-filling member is a single piece of material.

8. The dressing of claim 1, wherein the wound-filling member is made of polyurethane foam.

9. The dressing of claim 1, wherein the wound-filling member is made of a sponge-like material.

10. The dressing of claim 1, wherein the wound-filling member is made of a rubber-like material.

11. The dressing of claim 1, wherein the wound-filling member does not contain any active compounds.

12. The dressing of claim 1, wherein the wound-filling member has a 90% compression set of less than or equal to approximately 10%.

13. The dressing of claim 1, wherein the hemostasis inducer is impregnated with active compounds.

14. The dressing of claim 13, wherein the active compound comprises a coagulant.

15. The dressing of claim 14, wherein the active compound comprises kaolin.

16. The dressing of claim 1, wherein the wound-filling member is approximately 20 cm in width, approximately 20 cm in length and approximately 5 cm in thickness.

17. The dressing of claim 1, wherein the hemostasis inducer is approximately 30 cm in width and approximately 30 cm in length.

18. A dressing for treating wounds, the dressing comprising:
a hemostasis inducer with a wound contacting side and a back side;
a compressible member configured to conform to the shape of a wound cavity and coupled to the back side of the hemostasis inducer;
an elastic wrap attached to an edge of the hemostasis inducer;
a cover over the back side of the hemostasis inducer.

19. The dressing of claim 18, wherein the dressing is packaged in a compressed configuration.

20. The dressing of claim 18, wherein the compressible member is made of a foam-like material.

21. The dressing of claim 18, wherein the compressible member is a single piece of material.

22. The dressing of claim 18, wherein the compressible member is made of a sponge-like material.

23. The dressing of claim 18, wherein the compressible member is made of a rubber-like material.

24. The dressing of claim 18, wherein the compressible member does not contain any active compounds.

25. The dressing of claim 18, wherein the hemostasis inducer is impregnated with active compounds.

26. The dressing of claim 25, wherein the active compound comprises a coagulant.

27. The dressing of claim 26, wherein the active compound comprises kaolin.

28. A method of treating a wound, the method comprising the steps of:
opening a package containing a compressed wound dressing, the wound dressing comprising a hemostasis inducer with a wound contacting side and a back side, a compressible member attached to the back side of the gauze, and a wrap attached to an edge of the gauze;
placing the wound dressing in a cavity of the large wound, oriented such that the wound contacting side faces the cavity;
allowing the compressible member to expand at least partially within the cavity, wherein the compressible member at least partially presses the hemostasis inducer against the cavity;
wrapping the wrap around the hemostasis inducer and compressible member to secure the wound dressing to the wound.

29. The method of claim 28, wherein the hemostasis inducer is impregnated with a coagulant.
